(19) United States
(12) Patent Application Publication
(10) Pub. No.: US 2026/0284032 A1
(43) Pub. Date: Sep. 24, 2026

(54) METHODS OF TREATING DEPRESSION AND ANHEDONIA

(71) Applicant: Neumora Therapeutics, Inc., Watertown, MA (US)

(72) Inventor: Tien Dam, Lexington, MA (US)

(21) Appl. No.: 19/439,162

(22) Filed: Jan. 2, 2026

Related U.S. Application Data
(60) Provisional application No. 63/741,389, filed on Jan. 2, 2025.

(51) Int. Cl. A61K 31/4709; A61P 25/22; A61P 25/24

(52) U.S. Cl. CPC A61K 31/4709; A61P 25/22; A61P 25/24

(57) ABSTRACT

The present disclosure relates to methods of treating psychiatric disorders such as depression and anhedonia with a kappa opioid receptor antagonist.

### CLAIM OF PRIORITY

This application claims the benefit of U.S. Provisional Application Ser. No. 63/741,389, filed on Jan. 2, 2025. The entire contents of the foregoing are incorporated herein by reference.

### FIELD

The present disclosure relates to methods for treating psychiatric disorders such as anhedonia associated with depression.

### BACKGROUND

Depression is a leading cause of disability worldwide and a significant public health problem, particularly given the associated increased risk for other health comorbidities. See WHO (World Health Organ.) 2017. Depression and Other Common Mental Disorders: Global Health Estimates. Geneva: WHO and Greenberg, et al. J. Clin. Psychiatry 76:155-62 (2010). It frequently appears early in life, can occur chronically throughout life, and can adversely affect the prognosis of other medical illnesses such as cardiovascular and neurological conditions.

Anhedonia—the loss of pleasure or lack of reactivity to pleasurable stimuli—remains a significant treatment challenge. In subjects with major depressive disorder (MDD), for example, anhedonia has been linked to poor outcomes, such as a reduced response to psychological and psychiatric interventions, and an increased suicide risk. See, e.g., Pizzagalli, Am. J. Psychiatry, Vol. 179, No. 7, pp. 458-469 (2022).

While antidepressant medications and cognitive behavioral therapy can be effective for some individuals, up to 20% do not respond to these interventions, and many of those who do respond, eventually relapse. Similarly, an estimated 50% of depressed individuals are only partially (inadequately) treated by available clinical interventions. See Al Harbi, Patient Prefer. Adherence, Vol. 6, pp. 369-388 (2012) and Gorwood, Dialogues Clin. Neurocsci., Vol. 10, No. 3, pp. 291-299 (2008). In the NIMH Sequenced Treatment Alternatives to Relieve Depression (STAR*D) study, approximately half of patients treated with a first-line antidepressant therapy had reduction of symptoms to at least half of the original intensity and only approximately one-third of patients achieved remission (Chan 2013). While these patients may eventually recover, many require a trial and error approach to therapy, and many will ultimately develop treatment resistant depression (TRD) over time, i.e., failure to adequately respond to two or more courses of antidepressant treatment, and/or may still exhibit lingering anhedonia. See, e.g., Sackheim, J. Clin. Psychiatry, Vol. 62, Suppl. 16, pp. 10-17 (2001) and Fava M., Biol. Psychiatry 53:649-59 (2003) and McIntyre, et al., J. Affect. Disord. 156:1-7 (2014). This can lead to even more serious conditions such as suicidal ideation and suicidality.

The lack of efficacy and the significant adverse events associated with the use of current antidepressants leads to high levels of treatment discontinuation (Zajecka 2000). Even with multiple consecutive treatments, only a small proportion of patients remain asymptomatic (Rush 2007). In the meantime, individuals continue to suffer from anhedonia, have a risk of self-harm, and experience a negative impact in their personal and professional lives. See, e.g., Burcusa and Iacono, Clin. Psychol. Rev. Vol. 27, No. 8, pp. 959-985 (2007). Thus, methods to treat anhedonia would have a substantial impact on public health.

### SUMMARY

The details of one or more embodiments are set forth in the description below. Other features, objects, and advantages of the invention will be apparent from the description and from the claims.

Some embodiments provide a method of treating depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline Montgomery-Åsberg Depression Rating Scale (MADRS) total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe major depressive disorder (MDD).

Some embodiments provide a method of treating depression in a subject previously identified or diagnosed as having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject in need thereof has been previously identified or diagnosed as having anhedonia. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating depression in a female subject in need thereof, comprising:
(a) identifying a subject as having one or more symptoms of anhedonia; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating depression in a female subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating depression in a subject previously identified or diagnosed as having anhedonia, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating depression in a female subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject in need thereof has been previously identified or diagnosed as having anhedonia. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating depression in a female subject in need thereof, consisting essentially of:
(a) identifying a subject as having one or more symptoms of anhedonia; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating severe depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a MADRS total Score of from 25 to 60 (e.g., 25 to 34). In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating severe depression in a female subject in need thereof, consisting of administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a MADRS total score of from 25 to 60 (e.g., 25 to 34). In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating depression in a female subject in need thereof, comprising:
(a) determining a first MADRS Total Score in the subject;
(b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and
(c) determining a second MADRS Total Score in the subject; wherein the second MADRS total score is less than the first MADRS total score. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating depression in a female subject in need thereof, consisting of:
(a) determining a first MADRS total score in the subject;
(b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and
(c) determining a second MADRS total score in the subject; wherein the second MADRS total score is less than the first MADRS total score. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating depression in a female subject in need thereof, comprising:
(a) determining a first MADRS total score in the subject;
(b) determining a first Snaith-Hamilton Pleasure Scale (SHAPS) total score in the subject;
(c) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and
(d) determining a second MADRS total score in the subject;
(e) determining a second SHAPS total score in the subject;
wherein the second MADRS total score is less than the first MADRS total score and/or the second SHAPS total score is less than the first SHAPS total score. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating depression in a female subject in need thereof, consisting of:
(a) determining a first MADRS total score in the subject;
(b) determining a first SHAPS total score in the subject;
(c) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and
(d) determining a second MADRS total score in the subject;
(e) determining a second SHAPS total score in the subject;
wherein the second MADRS total score is less than the first MADRS total score and/or the second SHAPS total score is less than the first SHAPS total score. In some embodiments, the subject in need thereof has been previously identified or diagnosed as having moderate-to-severe anxiety. In some embodiments, the subject has a baseline MADRS total score ≥17. In some embodiments, a subject with a baseline MADRS total score ≥17 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating depression and anhedonia in a female subject in need thereof, consisting of administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating severe depression and severe anhedonia in a female subject in need thereof, consisting of administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating depression in a subject previously identified or diagnosed as having anhedonia, consisting of administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating severe depression in a subject previously identified or diagnosed as having severe anhedonia, consisting of administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of treating severe depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a MADRS total score of from 25 to 60 (e.g., 25 to 34). In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of reducing the severity of depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of reducing the severity of depression in a subject previously identified or diagnosed as having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of reducing the severity of depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject in need thereof has been previously identified or diagnosed as having anhedonia. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of reducing the severity of depression in a female subject in need thereof, comprising:
(a) identifying a subject as having one or more symptoms of anhedonia; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of reducing the severity of depression in a female subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of reducing the severity of depression in a subject previously identified or diagnosed as having anhedonia, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of reducing the severity of depression in a female subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject in need thereof has been previously identified or diagnosed as having anhedonia. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of reducing the severity of depression in a female subject in need thereof, consisting essentially of:
(a) identifying a subject as having one or more symptoms of anhedonia; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of reducing the severity of depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a MADRS total score of from 25 to 60 (e.g., 25 to 34). In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of reducing the severity of depression in a female subject in need thereof, comprising:
(a) determining a first MADRS total score in the subject;
(b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and
(c) determining a second MADRS total score in the subject; wherein the second MADRS total score is less than the first MADRS total score. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of reducing the MADRS total score in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of reducing the MADRS total score in a subject having depression, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

Some embodiments provide a method of reducing the MADRS total score in a subject previously identified or diagnosed as having depression, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

In some embodiments, the subject has a baseline MADRS total score 25 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has a MADRS total score of about 25 to 60 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has a MADRS total score of about 25 to 34 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has a MADRS total score of ≥25 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject is an adult female. In some embodiments, the subject is an adult female previously identified or diagnosed as having moderate-to-severe anxiety. For example, the subject can be an adult female previously identified or diagnosed as having moderate-to-severe MDD.

In some embodiments, the MADRS total score of the subject is decreased after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the MADRS total score of the subject is decreased after six weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

### DESCRIPTION OF THE FIGURES

The following drawings illustrate certain embodiments of the features and advantages of this disclosure. These embodiments are not intended to limit the scope of the appended claims in any manner.

FIG. 1 is a graph showing forest plot of subgroup analysis for Montgomery-Åsberg Depression Rating Scale (MADRS) total score change from baseline to week 6 for the study described in Example 1.

FIGS. 2A and 2B provide graphs describing MADRS total score change from baseline in female and male participants for the study described in Example 1.

FIG. 3 is a graph showing forest plot of subgroup analysis for Snaith-Hamilton Pleasure Scale (SHAPS) total score change from baseline to week 6 for the study described in Example 1.

FIGS. 4A and 4B provide graphs describing SHAPS total score change from baseline in female and male participants for the study described in Example 1.

### DETAILED DESCRIPTION

### Definitions

To facilitate understanding of the disclosure set forth herein, a number of additional terms are defined below. Generally, the nomenclature used herein and the laboratory and clinical procedures in organic chemistry, medicinal chemistry, pharmacology, and psychiatry described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The compound 1-(6-ethyl-8-fluoro-4-methyl-3-(3-methyl-1,2,4-oxadiazol-5-yl)quinolin-2-yl)-N-(tetrahydro-2H-pyran-4-yl)piperidin-4 amine, or a pharmaceutically acceptable salt thereof, is a kappa opioid receptor antagonist having the structure:

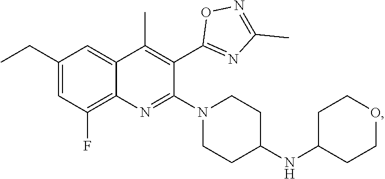 [SMILES: CCc1cc(F)c2nc(N3CCC(NC4CCOCC4)CC3)c(-c3nc(C)no3)c(C)c2c1]

or a pharmaceutically acceptable salt thereof described in, for example, U.S. Pat. No. 9,682,966.

The term “about” when referring to a number or a numerical range means that the number or numerical range referred to is an approximation, for example, within experimental variability and/or statistical experimental error, and thus the number or numerical range may vary up to 10% of the stated number or numerical range.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid; organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

As used herein, the “subject” refers to a human female, and “subject” and “female subject” are used herein interchangeably. In some embodiments, the subject has experienced and/or exhibited at least one symptom of the disorder to be treated. In some embodiments, the subject is an adult subject, e.g., an adult female subject. In some embodiments, the subject is previously identified or diagnosed as having moderate-to-severe major depressive disorder (MDD). For example, as used herein, a subject can be a female subject (who is previously identified or diagnosed as having moderate-to-severe MDD.

“Treatment” or “therapy” of a subject refers to any type of intervention or process performed on, or the administration of an active agent to, the subject with the objective of reversing, alleviating, ameliorating, inhibiting, or slowing down, the onset, progression, development, severity, or recurrence of a symptom, complication, condition, or biochemical indicia associated with a disease, in whole or in part. In some embodiments, “treatment” includes resolution of a particular disorder, including a reduction in one or more symptoms of the disorder and/or a reduction in in the severity of one or more symptoms associated with the disorder.

Unless indicated otherwise, a “score” and a “total score” are used interchangeably herein, for example, MADRS “Total Score” and MADRS “Score.”

“Administering” or “administration” refer to the physical introduction of a therapeutic agent to a subject, using any of the various methods and delivery systems known to those skilled in the art. Routes of administration can include, for example, oral or intravenous administration. Administration can also be performed, for example, once, a plurality of times, and/or over one or more extended periods.

A “therapeutically effective amount” of a therapeutic agent is any amount of the agent that, when used alone or in combination with one or more additional therapies, slows down the onset of a psychiatric disorder or promotes regression of the disorder evidenced by a decrease in severity of disorder symptoms, an increase in frequency and duration of disorder symptom-free periods, or a ameliorating an impairment or disability due to the disorder affliction (i.e., an amount sufficient to treat (as defined herein) the disorder). For example, a “therapeutically effective amount” of a therapeutic agent may result in amelioration, reduction, or elimination of at least one of the following symptoms: persistent sadness or anxiety, feelings of emptiness, hopelessness, pessimism, guilt, worthlessness, helplessness, a loss of interest or pleasure in hobbies and activities that were once enjoyed (anhedonia), decreased energy, fatigue, difficulty concentrating, remembering, or making decisions, insomnia, early-morning awakening, oversleeping, appetite loss, weight loss, overeating, weight gain, restlessness, irritability, and persistent physical symptoms that do not respond to treatment, such as headaches, digestive disorders, and chronic pain.

As used herein, a measure of a treatment effect is “clinically meaningful” based on the practical importance of a treatment effect. For example, whether the treatment effect has a real genuine, palpable, and/or noticeable effect on the subject (e.g., a lack of clinically meaningful effect occurs when the difference in the subject is small enough that it may be considered similar, such as prior to and after administration of a treatment as provided herein). One skilled in the art would recognize whether a particular effect is “clinically meaningful.” For example, a subject having a baseline score indicating severe depression (using any of the scales described herein) and a post-treatment score indicating remission of the severe depression would be a clinically meaningful effect.

A subject that is “not responsive” refers to a subject that has been, or is currently being, treated with one or more therapies that are not providing a clinically meaningful change towards the desired outcome (e.g., subjects that are not responsive includes patients that are refractory to a particular treatment). For example, a subject may exhibit no measurable change in response to therapy. A non-responsive subject could also, for example, exhibit a positive change in a depression scale score, but the change is not clinically meaningful.

As used herein, the “response rate” refers to the percentage of subjects that exhibit a clinically meaningful response to treatment with a particular agent, or combination of agents.

As used herein, a psychiatric evaluation or side effect profile test score that is “substantially similar” or “substantially the same” as a reference score, corresponds to the same score, with a skilled artisan understanding that particular test scores may vary to a reasonable extent (such as ±10%) while still describing a given value, due to, for example, experimental error, routine subject-to-subject evaluation, and routine statistical analysis.

The essential features of a “major depressive episode” or “major depression” is a period of at least 2 weeks during which there is either a depressed mood or the loss of interest or pleasure in nearly all activities. The individual must also experience at least four additional symptoms drawn from a list that includes changes in appetite or weight, sleep, and psychomotor activity; decreased energy; feelings of worthlessness or guilt; difficulty thinking, concentrating, or making decisions; or recurrent thoughts of death or suicidal ideation, plans or attempts. To be considered a major depressive episode, a symptom must have clearly worsened compared with the person's pre-episode status. The symptoms must persist for most of the day, nearly every day, for at least 2 consecutive weeks. The episode must be accompanied by clinically significant distress or impairment in social, occupational or other important areas of functioning (Diagnostic and Statistical Manual of Mental Disorders 4th Ed. DSM-IV, Pub. American Psychiatric Association, Washington, D.C.; p. 320, 327, 344-345). A “major depressive disorder” generally refers to a single or recurrent Major Depressive Episode which is not better accounted for by Schizophrenia, Delusional Disorder, or Psychotic Disorder Not Otherwise Specified, and also there has never been a Manic Episode, a Mixed Episode or a Hypomanic Episode (Diagnostic and Statistical Manual of Mental Disorders 4th Ed. DSM-IV, Pub. American Psychiatric Association, Washington, D.C.; pp. 344-345). The diagnosis of depression is generally based on evaluation by a qualified physician, generally a psychiatrist or by a psychologist. A “minor depressive disorder”, also referred to as “dysthymia”, has the characteristics of a major depressive disorder but presents itself without the intensity or severity of the symptoms associated with a “major depressive disorder”. “Late Life Major Depression”, referred to as “LLMD” or “late-onset depression” refers to depression, for example, the major and minor depressive disorders and depressive episodes described above, that occurs in a subject at about 60 years of age or older. The “risk factors” for depression include female gender, unmarried status, having stressful life events and lack of a social support network. Major depressive disorder is characterized by any of a number of symptoms, including persistent sadness or anxiety, or feelings of emptiness, hopelessness, pessimism, guilt, worthlessness, or helplessness.

As used in the methods described herein, the term “reducing” refers to a reduction in the indicated parameter relative to the baseline measurement (or measurements) of the same parameter in the subject taken prior to the initiation of administration or a kappa opioid receptor antagonist, or a reduction in the indicated parameter relative to the baseline measurement (or measurements) of the same parameter. In some embodiments, the same parameter is measured in a healthy subject (for example, a subject that does not have a psychiatric disorder as described herein). In some embodiments, the same parameter is measured relative to another treatment modality (for example, the standard of care treatment for a depression as described herein).

Similarly, the term “increasing,” as used herein, refers to an increase in the indicated parameter relative to the baseline measurement (or measurements) of the same parameter in the subject taken prior to the initiation of administration of a kappa opioid receptor antagonist, or an increase in the indicated parameter relative to the baseline measurement (or measurements) of the same parameter. In some embodiments, the same parameter is measured in a healthy subject (for example, a subject that does not have depression as described herein). In some embodiments, the same parameter is measured relative to another treatment modality (for example, the standard of care treatment for depression as described herein).

Measurements of certain parameters described herein can be qualitative (e.g., based on patient description of feelings) and/or quantitative (e.g., based on scale scores, as described herein). Subject feelings and/or behavior can be self-reported or assessed by a third party such as a family member, friend, physician, counselor, or other caregiver.

As described herein, any concentration range, percentage range, ratio range, or integer range is to be understood to include the value of any integer within the recited range and, when appropriate, fractions thereof (such as one tenth and one hundredth of an integer), unless otherwise indicated.

### Introduction

Depression is characterized by depressed mood and a markedly diminished interest or pleasure in activities, e.g., anhedonia. Other symptoms may include significant weight loss or weight gain, decrease or increase in appetite, insomnia or hypersomnia, psychomotor agitation or retardation, fatigue or loss of energy, feelings of worthlessness or excessive or inappropriate guilt, and a diminished ability to think or concentrate or indecisiveness. See Kennedy, Dialogues Clin. Neurosci., Vol. 10, No. 3, pp. 271-277 (2008). A variety of somatic symptoms may also be present. Though depressive feelings are common, depressive disorder is diagnosed only when the symptoms reach a threshold and last at least two weeks. Depression can vary in severity from mild to very severe. It is most often episodic but can be recurrent or chronic. More than 50% of those who initially suffer a single major depressive episode eventually develop another. Unfortunately, current pharmacological interventions for depression take weeks to months to achieve their full therapeutic effect, and many subjects are, or will become, resistant to these therapies. See, e.g., Kupfer, Dialogues Clin. Neurosci., Vol. 7, No. 3, pp. 191-205 (2005).

The presence of anhedonia is associated with difficulty in treating major depressive disorder. Research findings indicate that available therapies do not target depression-related motivational and reward-processing deficits sufficiently (APA 2000; Dunlop and Nemeroff 2007; McCabe et al., 2010; Nutt et al., 2007; Price et al., 2009; Shelton and Tomarken 2001) and that anhedonic symptoms predict inadequate treatment response (Spijker et al., 2001).

The presence of anhedonia is associated with inadequate treatment response to antidepressant drugs (McMakin et al., 2012; Uher et al., 2012) and potentially also to psychological treatments (Craske et al., 2016). Unfortunately, research findings indicate that available therapies such as SSRIs do not target depression-related motivational and reward-processing deficits sufficiently (APA 2000; Dunlop and Nemeroff 2007; McCabe et al., 2010; Nutt et al., 2007; Price et al., 2009; Shelton and Tomarken 2001).

Major depressive disorder (MDD) is the most common mood disorder and imposes considerable economic and humanitarian suffering, such as decreased quality of life, functional impairment, and increased mortality rate. Currently, MDD is the leading cause of disability and afflicts approximately 322 million people worldwide (4.4% of the global population), with prevalence increasing 18% between 2005 and 2015 (World Health Organization [WHO]2017). Depressive disorders were expected to be the second highest cause of morbidity in the world by 2020 (Murray and Lopez 2006) and have been predicted to become the leading cause of disease burden by the year 2030 (WHO 2004). The lifetime prevalence of MDD is approximately 16.6% in the United States (US) (Kessler et al., 2003).

Episodes of MDD are often chronic and recurrent, with a relapse chance rate of 55% to 90% for individuals who experienced one or two prior depressions. More than 80% of the individuals who experience a second episode and who are not treated will experience a third episode within 3 years (Thase and Sullivan 1995).

A clinical diagnosis of MDD is made based on the continuous presence of at least 5 of 9 symptoms over at least 2 weeks. One of these symptoms, as defined in the Diagnostic and Statistical Manual of Mental Disorders, 5th Edition (DSM-5), must be either depressed mood or anhedonia, which is defined as diminished interest or pleasure in response to rewarding stimuli (APA 2013). Indeed, a significant number of subjects with depression do not achieve a sustained and complete response even after multiple therapeutic trials. See, e.g., Rush, et al., Psychol. Med. Vol. 52, pp. 419-432 (2022), which is incorporated by reference in its entirety.

The kappa opioid receptor (KOR) is a seven transmembrane-spanning G protein-coupled receptor encoded by the opioid receptor kappa 1 (OPRK1) gene. It functions as a receptor for endogenous ligands, as well as a receptor for various synthetic opioids. KOR plays a role in the perception of pain and mediating the hypolocomotor, analgesic and aversive actions of synthetic opioids. See e.g., Lalanne et al., Front Psychiatry (2014) 5:170.

Activation of kappa opioid receptors (KORs) produces negative affect. For example, KOR agonists produce dysphoric effects and elicit psychotomimetic properties in humans, as well as elicit place aversion and depressive-like affective behaviors in rodents. See Chavkin and Koob, (2016) Neuropsychopharmacology 41:373-374. One mechanism implicated in x-mediated aversion is the modulation of mesolimbic dopamine circuitry, where KORs are expressed on dopamine terminals. Activation of KORs following systemic agonist treatment reduces dopamine release. See Chefer et al., Neuropsychopharmacology (2013) 38:2623-2631. Ablation of KORs from dopamine neurons KORs on BLA glutamatergic neurons that project to the medial PFC results in an anxiolytic phenotype, suggesting that these circuits are critical to the expression of negative affective-like behavior.

The present application is based, in part, on the surprising discovery that while certain kappa opioid receptor antagonists are unable to exert a clinically meaningful impact in subjects having depression and anhedonia, administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof provides a clinically meaningful reduction in symptoms of depression and anhedonia and a concomitant increase in quality of life for those subjects. See, e.g., U.S. Pat. No. 11,266,627 (col. 38-41), and clinical trial results for 2019-000695-41 available on the European Union Clinical Trials Register (www.clinicaltrialsregister.eu/ctr-search/trial/2019-000695-41/results), which are hereby incorporated by reference in their entirety for the limited purpose of comparative data.

### Methods of Treatment

Some embodiments provide a method of treating depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline Montgomery-Åsberg Depression Rating Scale (MADRS) total score 25. In some embodiments, a subject with a baseline MADRS total score 25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of treating depression in a subject previously identified or diagnosed as having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of treating depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject in need thereof has been previously identified or diagnosed as having anhedonia. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of treating depression in a female subject in need thereof, comprising:
(a) identifying a subject as having one or more symptoms of anhedonia; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of treating depression in a female subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety.

Some embodiments provide a method of treating depression in a subject previously identified or diagnosed as having anhedonia, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety.

Some embodiments provide a method of treating depression in a female subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject in need thereof has been previously identified or diagnosed as having anhedonia. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety.

Some embodiments provide a method of treating depression in a female subject in need thereof, consisting essentially of:
(a) identifying a subject as having one or more symptoms of anhedonia; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety.

Some embodiments provide a method of treating severe depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a MADRS total score of from 25 to 60 (e.g., from 25 to 34). In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of treating severe depression in a female subject in need thereof, consisting of administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a MADRS total score of from 25 to 60 (e.g., from 25 to 34). In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety.

Some embodiments provide a method of treating depression and anhedonia in a female subject in need thereof, consisting of administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety.

Some embodiments provide a method of treating severe depression and severe anhedonia in a female subject in need thereof, consisting of administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety.

Some embodiments provide a method of treating depression in a subject previously identified or diagnosed as having anhedonia, consisting of administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety.

Some embodiments provide a method of treating severe depression in a subject previously identified or diagnosed as having severe anhedonia, consisting of administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety.

Some embodiments provide a method of reducing the severity of depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the severity of depression in a subject previously identified or diagnosed as having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the severity of depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject in need thereof has been previously identified or diagnosed as having anhedonia. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the severity of depression in a female subject in need thereof, comprising:
(a) identifying a subject as having one or more symptoms of anhedonia; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety. In some embodiments, the method further comprises determining a baseline MADRS total score in the subject before administering 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the severity of depression in a female subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety.

Some embodiments provide a method of reducing the severity of depression in a subject previously identified or diagnosed as having anhedonia, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety.

Some embodiments provide a method of reducing the severity of depression in a female subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject in need thereof has been previously identified or diagnosed as having anhedonia. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety.

Some embodiments provide a method of reducing the severity of depression in a female subject in need thereof, consisting essentially of:
(a) identifying a subject as having one or more symptoms of anhedonia; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject in need thereof has been previously identified or diagnosed as having moderate-to-severe anxiety. In some embodiments, the subject has a baseline MADRS total score ≥25. In some embodiments, a subject with a baseline MADRS total score ≥25 is identified or diagnosed as having moderate-to-severe anxiety.

Some embodiments provide a method of reducing the severity of depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a MADRS total score of from 25 to 60. For example, some embodiments provide a method of reducing the severity of depression in a female subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a MADRS total score of from 25 to 34, or from 35 to 60.

Some embodiments provide a method of reducing the severity of depression in a female subject in need thereof, comprising: (a) determining a first MADRS total score in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and (c) determining a second MADRS total score in the subject; wherein the second MADRS total score is less than the first MADRS total score. In some embodiments, the second MADRS total score is less than the first MADRS total score after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the second MADRS total score is less than the first MADRS total score after six weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the MADRS total score in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. For example, some embodiments provide a method of reducing the MADRS total score in a female subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the MADRS total score of the subject is decreased after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. Additionally, or in the alternative, some embodiments provide a method of reducing the MADRS total score in a female subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the MADRS total score of the subject is decreased after six weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the MADRS total score in a subject having depression, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. For example, some embodiments provide a method of reducing the MADRS total score in a subject having depression, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the MADRS total score of the subject is decreased after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. Additionally, or in the alternative, some embodiments provide a method of reducing the MADRS total score in a subject having depression, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the MADRS total score of the subject is decreased after six weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the MADRS total score in a subject previously identified or diagnosed as having depression, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. For example, some embodiments provide a method of reducing the MADRS total score in a subject previously identified or diagnosed as having depression, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof; wherein the MADRS total score of the subject is decreased after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. Additionally, or in the alternative, some embodiments provide a method of reducing the MADRS total score in a subject previously identified or diagnosed as having depression, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof; wherein the MADRS total score of the subject is decreased after six weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the Snaith-Hamilton Pleasure Scale (SHAPS) Total Score in a subject having depression, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof; wherein the subject has a SHAPS Total Score of 31 to 56 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. For example, some embodiments provide a method of reducing the SHAPS Total Score in a subject having depression, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine or the pharmaceutically acceptable salt thereof, the subject has a SHAPS Total Score of 33 to 56, 35 to 56, 37 to 56, 39 to 56, 41 to 56, 43 to 56, 45 to 56, 47 to 56, 49 to 56, 51 to 56, or 53 to 56.

Some embodiments provide a method of reducing the SHAPS Total Score in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. For example, some embodiments provide a method of reducing the SHAPS Total Score in a female subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine or the pharmaceutically acceptable salt thereof, the SHAPS Total Score of the subject is decreased by about 2 to about 4, or by about 3. Additionally, or in the alternative, some embodiments provide a method of reducing the SHAPS Total Score in a female subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine or the pharmaceutically acceptable salt thereof, the SHAPS Total Score of the subject is decreased by about 4 to about 6, or by about 5.

Some embodiments provide a method of reducing the SHAPS Total Score in a subject having depression, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. For example, some embodiments provide a method of reducing the SHAPS Total Score in a subject having depression, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine or the pharmaceutically acceptable salt thereof, the SHAPS Total Score of the subject is decreased by about 2 to about 4, or by about 3. Additionally, or in the alternative, some embodiments provide a method of reducing the SHAPS Total Score in a subject having depression, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine or the pharmaceutically acceptable salt thereof, the SHAPS Total Score of the subject is decreased by about 4 to about 6, or by about 5.

Some embodiments provide a method of reducing the SHAPS Total Score in a subject previously identified or diagnosed as having depression, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. For example, some embodiments provide a method of reducing the SHAPS Total Score in a subject previously identified or diagnosed as having depression, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof; wherein after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine or the pharmaceutically acceptable salt thereof, the SHAPS Total Score of the subject is decreased by about 2 to about 4, or by about 3. Additionally, or in the alternative, some embodiments provide a method of reducing the SHAPS Total Score in a subject previously identified or diagnosed as having depression, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine or the pharmaceutically acceptable salt thereof, the SHAPS Total Score of the subject is decreased by about 4 to about 6, or by about 5.

In some embodiments, the subject is an adult female. In some embodiments, the subject is an adult female previously identified or diagnosed as having moderate-to-severe anxiety. In some embodiments, the subject is an adult female previously identified or diagnosed as having moderate-to-severe MDD.

In some embodiments, the depression is major depressive disorder (MDD) or treatment resistant depression (TRD).

In some embodiments, the depression is major depressive disorder (MDD).

In some embodiments, the depression is treatment resistant depression (TRD). Treatment resistant depression, as described herein, includes depression that does not adequately respond to a course of treatment for depression within a defined time period (e.g., as defined by a clinician). An inadequate response includes, but is not limited to, less than an 80% reduction in depressive symptoms, less than a 75% reduction in depressive symptoms, less than a 70% reduction in depressive symptoms, less than a 65% reduction in depressive symptoms, less than a 60% reduction in depressive symptoms, less than a 55% reduction in depressive symptoms, less than a 50% reduction in depressive symptoms, less than a 45% reduction in depressive symptoms, less than a 40% reduction in depressive symptoms, less than a 35% reduction in depressive symptoms, or less than a 30% reduction in depressive symptoms. In some embodiments, the subject fails to adequately respond to two or more courses of antidepressant treatment, such as two, three, four, of five courses of treatment.

In some embodiments, the depression of a subject demonstrates a partial response, inadequate response, or residual symptomatology following treatment.

In some embodiments, the depression is difficult-to-treat depression (DTD).

Potential parameters that can be used, for example, to define whether a subject has DTD and/or characterize a subject within a sub-group of DTD include, but are not limited to early life trauma, concurrent medications, functional impairment, family history, variability of symptoms, adherence to treatment protocols, symptom features (such as anhedonia), the course of depression, cormorbid psychiatric conditions or other general medical conditions, the number and sequence of failed treatments, and the types of failed treatment (and types of failure, such as non-responsiveness or non-compliance due to side effects). Subjects with DTD may be non-responsive to current antidepressant therapies, or may exhibit an initial response that wanes over time, ultimately with the depressive symptoms returning despite continuing treatment. Accordingly, in some embodiments, the methods of treating DTD described herein further comprise additional clinical evaluation of (i) the durability of benefit of treatment, (ii) the side effect burden of treatment, if any, and/or (iii) presence of a sustained impact on quality of life and/or or daily function. Such additional clinical evaluations can occur before and/or during treatment, and can be conducted by a clinician and/or self-reported by the subject.

In some embodiments, the depression further comprises anxiety.

In some embodiments, the subject maintains about the same weight before administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof and after administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time. In some embodiments, this period of time is, for example, about 1 month, about 2 months, about 3 months, about 4, months, about 5 months, about 6 months, about 7 months, about 8 months, about 9 months, about 10 months, about 11 months, about 1 year, about 2 years, about 3 years, about 4, years, about 5 years, about 6 years, about 7 years, about 8 years, about 9 years, or about 10 years or more.

In some embodiments, the subject maintains about the level of sexual function before administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof and after administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time. In some embodiments, this period of time is, for example, about 1 month, about 2 months, about 3 months, about 4, months, about 5 months, about 6 months, about 7 months, about 8 months, about 9 months, about 10 months, about 11 months, about 1 year, about 2 years, about 3 years, about 4, years, about 5 years, about 6 years, about 7 years, about 8 years, about 9 years, or about 10 years or more. The level of sexual function can be assessed, for example, by clinical evaluation, self-reporting, and/or reporting from the subject's partner(s).

In some embodiments, the subject does not experience significant weight gain during treatment with 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. Significant weight gain refers to a 10% or higher increase in body weight.

In some embodiments, the subject does not experience sexual dysfunction during treatment with 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the method comprises a reduction in one or more depressive symptoms in the subject. Depressive symptoms include, but are not limited to dysthymia, feelings of sadness, tearfulness, emptiness or hopelessness; angry outbursts, irritability and/or frustration, even over small matters; loss of interest or pleasure in most or all normal activities; sleep disturbances; insomnia; sleeping too much; tiredness and/or lack of energy, reduced appetite and/or weight loss; increased cravings for food; weight gain; anxiety; agitation; restlessness; slowed thinking; slowed speaking; slowed body movements; feelings of worthlessness; feelings of guilt; fixating on past failures; self-blame; trouble thinking, concentrating, making decisions, and/or remembering things; frequent and/or recurrent thoughts of death, suicidal thoughts, suicide attempts, or suicide; and unexplained physical problems, such as back pain and/or other chronic pain, digestive disorders, and/or headaches.

In some embodiments, the subject has a SHAPS Total Score of from 31 to 56 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the subject has a SHAPS Total Score of from 37 to 56 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the subject has a SHAPS Total Score of from 31 to 37 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, the subject has a SHAPS Total Score of from 31 to 56, 31 to 50, 31 to 44, 31 to 37, 37 to 56, 43 to 56, or 50 to 56.

In some embodiments, the subject has been previously identified as having a SHAPS Total Score of from 31 to 56, 31 to 50, 31 to 44, 31 to 37, 37 to 56, 43 to 56, or 50 to 56. In some embodiments, the subject has been previously identified as having a SHAPS Total Score of from 31 to 37.

In some embodiments, the first SHAPS Total Score is from 31 to 56, 31 to 50, 31 to 44, 31 to 37, 37 to 56, 43 to 56, or 50 to 56. In some embodiments, the first SHAPS Total Score is from 31 to 37. In some embodiments, the first SHAPS Total Score is from 31 to 56. In some embodiments, the first SHAPS Total Score is from 37 to 56.

In some embodiments, the second SHAPS Total Score is lower than the first SHAPS Total Score by about 2 to about 5. In some embodiments, the second SHAPS Total Score is lower than the first SHAPS Total Score by 2 to 5. In some embodiments, the second SHAPS Total Score is from 29 to 50. In some embodiments, the second SHAPS Total Score is from 26 to 47. In some embodiments, the second SHAPS Total Score is from 35 to 50. In some embodiments, the second SHAPS Total Score is from 32 to 47. In some embodiments, the second SHAPS Total Score is less than 29.

In some embodiments, the SHAPS Total Score of the subject is decreased by about 2 to about 4 after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS Total Score of the subject is 29 or less, or 27 or less, after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS Total Score of the subject is 35 or less, or 33 or less, after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS Total Score of the subject is decreased by about 3 after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS Total Score of the subject is from 27 to 29 after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS Total Score of the subject is from 33 to 35 after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS Total Score of the subject is decreased by about 4 to about 6 after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS Total Score of the subject is 27 or less, or 25 or less, after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS Total Score of the subject is 33 or less, or 31 or less, after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS Total Score of the subject is decreased by about 5 after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS Total Score of the subject is from 25 to 27 after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS Total Score of the subject is from 31 to 33 after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the subject has previously been administered one or more antidepressant medications. In some embodiments, the subject has previously been administered one, two, or three antidepressant medications. In some embodiments, the subject has previously been administered one or two antidepressant medications. In some embodiments, the subject has previously been administered one antidepressant medication.

In some embodiments, the subject did not exhibit a clinically meaningful response to the one or more previously administered antidepressant medications.

In some embodiments, the one or more previously administered antidepressant medications are selected from selective serotonin reuptake inhibitors, selective norepinephrine reuptake inhibitors, and kappa opioid receptor antagonists.

In some embodiments, the one or more previously administered antidepressant medications are selected from selective serotonin reuptake inhibitors and selective norepinephrine reuptake inhibitors.

In some embodiments, the subject has been previously administered one or more selective serotonin reuptake inhibitors. In some embodiments, the subject has been previously administered one or more selective norepinephrine reuptake inhibitors.

In some embodiments, the subject has been previously administered one or more kappa opioid receptor antagonists. In some embodiments, the kappa opioid receptor antagonist is aticaprant (4-(4-(((2S)-2-(3,5-dimethylphenyl)-1-pyrrolidinyl)methyl)phenoxy)-3-fluorobenzamide).

Some embodiments provide a method of treating depression in a female subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject was previously administered aticaprant and discontinued treatment with aticaprant.

Some embodiments provide a method of treating depression in a subject previously administered aticaprant, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject did not exhibit a clinically meaningful response after administration of aticaprant for a period of time.

In some embodiments, the subject was previously administered about 5 mg to about 20 mg of aticaprant. In some embodiments, the subject was previously administered about 10 mg of aticaprant.

In some embodiments, the subject has not been previously administered one or more antidepressant medications. In some embodiments, the subject has not been previously administered a selective serotonin reuptake inhibitor. In some embodiments, the subject has not been previously administered a selective norepinephrine reuptake inhibitor. In some embodiments, the subject has not been previously administered a kappa opioid receptor antagonist.

In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is from about 20 mg/day to about 160 mg/day. For example, about 20 mg/day to about 80 mg/day, about 40 mg/day to about 100 mg/day, about 60 mg/day to about 120 mg/day, about 80 mg/day to about 140 mg/day, or about 100 mg/day to about 160 mg/day.

In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is from about 20 mg/day to about 80 mg/day. In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is from about 80 mg/day to about 120 mg/day. In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is from about 120 mg/day to about 160 mg/day.

In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is 40 mg/day. In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is 80 mg/day.

In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is 120 mg/day. In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is 160 mg/day.

In some embodiments, the 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is administered orally. In some embodiments, the 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is administered once per day.

In some embodiments, the subject has not had a score of “YES” on C-SSRS Item 4 or Item 5 within 3 months prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has not had a score of “YES” on C-SSRS Item 4 within 3 months prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has not had a score of “YES” on C-SSRS Item 5 within 3 months prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the 1-[6-ethyl-8-fluoro-4-methyl-3-(3-methyl-1,2,4-oxadiazol-5-yl)quinolin-2-yl]-N-(oxan-4-yl)piperidin-4-amine is administered as the free base.

In some embodiments, the 1-[6-ethyl-8-fluoro-4-methyl-3-(3-methyl-1,2,4-oxadiazol-5-yl)quinolin-2-yl]-N-(oxan-4-yl)piperidin-4-amine is administered as a pharmaceutically acceptable salt.

In some embodiments, the subject is an adult female previously identified or diagnosed as having moderate-to-severe anxiety. In some embodiments, the subject is an adult female previously identified or diagnosed as having moderate-to-severe MDD.

Measuring Depression and/or Anhedonia

Many methods can be used to measure depression and/or anhedonia in a subject. Non-limiting examples are described herein. When a scale score or change of score is described, for example, the scales discussed below, it can be the score or change of score for a particular subject (e.g., such a score would typically be an integer) or an average of the score of two or more subjects (e.g., a score that could be an integer or between integers).

### Hamilton Depression Rating Scale 17

The Hamilton Depression Rating Scale 17 (also referred to as HAMD, HRSD, HDRS, or HAMD-17) is a 17-item version of a 21-item semi-structured clinician-administered interview scale used to assess severity of, and change in, depressive symptoms in an adult patient diagnosed as suffering from depression. See, e.g., Hamilton, J. Neurol. Neurosurg. Psychiatry. 1960 Feb. 23:56-62 (1960); and Williams, Eur. Arch. Psychiatry. Clin. Neurosci. 251 (suppl 2):116 (2001).

The 17-item version focuses more on somatic symptoms than on cognitive or affective symptoms, and includes: 1) depressed mood; 2) guilt; 3) suicide; 4) insomnia—initial; 5) insomnia—middle; 6) insomnia—delayed; 7) work and interests; 8) psychomotor retardation; 9) agitation; 10) psychic anxiety; 11) somatic anxiety; 12) gastrointestinal somatic symptoms; 13) general somatic symptoms; 14) genital somatic symptoms; 15) hypochondriasis; 16) insight; 17) loss of weight. Each item either is graded on a 5-point scale (0-4), with a score of (0) representing absent; (1), mild; (2, 3), moderate; or (4), severe symptoms; or, on a 3-point scale (0-2), with a score of (0) representing absent; (1), slight or doubtful; and (2), clearly present symptoms. The 17 items are added to provide a total score, with a maximum score of 52. A higher score is indicative of more severe symptoms, for example, HAM-D score level of depression of 0-7 is considered not depressed; 8-13 is considered subthreshold to mild; 14-18 is considered mild to moderate; 19-22 is considered moderate to severe; and ≥23 is considered severe to very severe.

The additional four items on the 21-item scale are diurnal variation, depersonalization/derealization, paranoid symptoms, and obsessional and compulsive symptoms. These criteria are generally not used as they may be less likely due to the disease, they were infrequent, and/or they are not considered markers of disease severity. See, e.g., Miller et al., Psych. Res. 14:131-142 (1984) and Hamilton, Br. J. Soc. Clin. Psychol. 6 (4): 278-96 (1967). There is also an abbreviated 6 item version (HAMD-6) derived from HAMD-17 and scoring the following 6 items from HAMD-17: item 1—depressed mood; item 2—guilt; item 7—work and interest; item 8—psychomotor retardation; item 10—psychic anxiety; and item 13—general somatic symptoms. The HAMD-6 Total Score can be up to 22, with a higher score indicating more severe symptoms.

### Clinical Global Impression of Improvement (CGI-I)

Clinical Global Impression of Improvement (CGI-I) score is a subscale of Clinical Global Impression (CGI), a retrospective assessment that is a measure of symptom severity, treatment response and the efficacy of treatments in treatment studies, completed by the treating physician at baseline and at subsequent clinic visits to document any change in target symptoms documented at baseline. Thus, CGI-I provides overall comparison of the patient's baseline condition with his current state.

Each time the patient is seen after medication has been initiated, the clinician compares the patient's overall clinical condition to the one week period just prior to the initiation of medication use (the so-called baseline visit), and the following one query only is rated on the 7-point scale: Compared to the patient's condition at admission to the project [prior to medication initiation], this patient's condition is: 1=very much improved since the initiation of treatment (nearly all better; good level of functioning; minimal symptoms; represents a very substantial change); 2=much improved (notably better with significant reduction of symptoms; increase in the level of functioning but some symptoms remain); 3=minimally improved (slightly better with little or no clinically meaningful reduction of symptoms. Represents very little change in basic clinical status, level of care, or functional capacity); 4=no change from baseline (the initiation of treatment) (symptoms remain essentially unchanged); 5=minimally worse (slightly worse but may not be clinically meaningful; may represent very little change in basic clinical status or functional capacity); 6=much worse (clinically significant increase in symptoms and diminished functioning); 7=very much worse since the initiation of treatment (severe exacerbation of symptoms and loss of functioning). See Busner and Targum, Psychiatry, 4(7), 28-37 (2007).

### Snaith-Hamilton Pleasure Scale (SHAPS)

The Snaith-Hamilton Pleasure Scale (SHAPS) score is a 14-item self-administered questionnaire used to measure hedonic capacity. The subjects indicate whether they experience pleasure in performing particular activities or experiences related to social interaction, food and drink, sensory experience, and interest/pastimes.

Specifically, the 14 items include: (1) I would enjoy my favorite television or radio program; (2) I would enjoy being with my family or close friends; (3) I would find pleasure in my hobbies and pastimes; (4) I would be able to enjoy my favorite meal; (5) I would enjoy a warm bath or refreshing shower; (6) I would find pleasure in the scent of flowers or the smell of a fresh sea breeze or freshly baked bread; (7) I would enjoy seeing other people's smiling faces; (8) I would enjoy looking smart when I have made an effort with my appearance; (9) I would enjoy reading a book, magazine or newspaper; (10) I would enjoy a cup of tea or coffee or my favorite drink; (11) I would find pleasure in small things, e.g. bright sunny day, a telephone call from a friend; (12) I would be able to enjoy a beautiful landscape or view; (13) I would get pleasure from helping others; (14) I would feel pleasure when I receive praise from other people.

Each of the items has four response categories: Definitely/Strongly Agree; Agree; Disagree; and Definitely/Strongly Disagree, which are scored as a 4, 3, 2, or 1, respectively. Thus, the SHAPS are scored as the sum of the 14 items so that total scores ranged from 14 to 56. A higher total SHAPS Total Score indicates higher levels of current anhedonia. See, e.g., Snaith, et al., Br. J. Psychiatry, 167(1), 99-103 (1995) and Snaith, Psychol. Med. 23: 957-966 (1993).

### Hospital Anxiety and Depression Scale (HADS)

The Hospital Anxiety and Depression Scale (HADS) is a self-assessment mood scale that measures anxiety and depression. The HADS includes two conjoint 7-item subscales, one specifically targeted at anxiety (HADS-A) and one focusing on depression (HADS-D). HADS excludes many somatic symptoms for example dizziness and sleep disturbance although does include, for example, psychomotor agitation. The depression scale focuses on anhedonia. See, e.g., Zigmond and Snaith, Acta Psychiatrica Scandinavica, 67: 361-370 (1983) and Stern, Occupat. Med., 64(5): 393-394 (2014).

The 14 items include: (1) I feel tense or wound up; (2) I get a sort of frightened feeling as if something awful is about to happen; (3) Worrying thoughts go through my mind; (4) I can sit at ease and feel relaxed; (5) I get a sort of frightened feeling like ‘butterflies’ in the stomach; (6) I feel restless as I have to be on the move; (7) I get sudden feelings of panic; (8) I still enjoy the things I used to enjoy; (9) I can laugh and see the funny side of things; (10) I feel cheerful; (11) I feel as if I am slowed down; (12) I have lost interest in my appearance; (13) I look forward with enjoyment to things; (14) I can enjoy a good book or radio or TV program.

Each item is coded from 0 (no presence) to 3 (severe). The scores for anxiety and depression thus range from 0 to 21, with higher scores indicating more severe anxiety or depression. Scores of less than 7 indicate the subject is not anxious or depressed; Scores of 8-10 indicate mild anxiety or depression; 11-14=Scores of indicate moderate anxiety or depression; and Scores of 15-21 indicate severe anxiety or depression. See, e.g., Djukanovic, et al., Health Qual. Life Outcomes, 15(193) (2017) and Snaith, Health Qual. Life Outcomes, 1(29) (2003).

### Hamilton Anxiety Rating Scale (HAM-A) Score

The Hamilton Anxiety Rating Scale (HAM-A, HARS) score is a clinician-based questionnaire for measuring the severity of anxiety symptoms. The scale includes 14 items, each defined by a series of symptoms, and measures both psychic anxiety (mental agitation and psychological distress) and somatic anxiety (physical complaints related to anxiety). Specifically: (1) anxious mood; (2) tension (including startle response, fatigability, restlessness); (3) fears (including of the dark/strangers/crowds); (4) insomnia; (5) ‘intellectual’ (poor memory/difficulty concentrating); (6) depressed mood (including anhedonia); (7) somatic symptoms (including aches and pains, stiffness, bruxism); (8) sensory (including tinnitus, blurred vision); (9) cardiovascular (including tachycardia and palpitations); (10) respiratory (chest tightness, choking); (11) gastrointestinal (including irritable bowel syndrome-type symptoms); (12) genitourinary (including urinary frequency, loss of libido); (13) autonomic (including dry mouth, tension headache); and (14) observed behavior at interview (restless, fidgety, etc.). See, e.g., Hamilton, Br. J. Med. Psychol. 32:50-55 (1959) and Thompson, Occupat. Med., 65(7): 601, (2015).

Each item is scored from 0-4, referring to 0=not present; 1=mild degree; 2=moderate degree; 3=marked degree; 4=maximum degree, providing a total score range of 0-52, where higher score indicates greater symptom severity. A total score of 6-14 indicates mild anxiety; a total score of 15-28 indicates moderate anxiety; and a total score of 29-52 indicates severe anxiety.

In some embodiments, a HAM-A score of about 15 to 52, about 17 to 52, about 19 to 52, about 21 to 52, about 23 to 52, about 25 to 52, about 27 to 52, about 29 to 52, about 30 to 52, about 32 to 52, about 34 to 52, about 36 to 52, about 38 to 52, about 40 to 52, about 42 to 52, about 44 to 52, about 46 to 52, about 48 to 52, and/or about 50 to 52 indicates moderate-to-severe anxiety. For example, a subject with a HAM-A score (e.g., a baseline HAM-A score) of about 15 to 52, about 17 to 52, about 19 to 52, about 21 to 52, about 23 to 52, about 25 to 52, about 29 to 52, about 30 to 52, about 32 to 52, about 34 to 52, about 36 to 52, about 38 to 52, about 40 to 52, about 42 to 52, about 44 to 52, about 46 to 52, about 48 to 52, and/or about 50 to 52 can be identified or diagnosed as having moderate-to-severe anxiety. In some embodiments, a HAM-A score of about 15 to 28, about 17 to 28, about 19 to 28, about 21 to 28, about 23 to 28, about 25 to 28, and/or about 27 to 28 indicates moderate-to-severe anxiety. For example, a subject with a HAM-A score (e.g., a baseline HAM-A score) of about 15 to 28, about 17 to 28, about 19 to 28, about 21 to 28, about 23 to 28, about 25 to 28, and/or about 27 to 28 can be identified or diagnosed as having moderate-to-severe anxiety. In some embodiments, a HAM-A score of about 29 to 52, about 30 to 52, about 32 to 52, about 34 to 52, about 36 to 52, about 38 to 52, about 40 to 52, about 42 to 52, about 44 to 52, about 46 to 52, about 48 to 52, and/or about 50 to 52 indicates moderate-to-severe anxiety. For example, a subject with a HAM-A score (e.g., a baseline HAM-A score) of about 29 to 52, about 30 to 52, about 32 to 52, about 34 to 52, about 36 to 52, about 38 to 52, about 40 to 52, about 42 to 52, about 44 to 52, about 46 to 52, about 48 to 52, and/or about 50 to 52 can be identified or diagnosed as having moderate-to-severe anxiety.

In some embodiments, a HAM-A score ≥15, a HAM-A score ≥17, a HAM-A score ≥19, a HAM-A score ≥21, a HAM-A score ≥23, a HAM-A score ≥25, a HAM-A score ≥27, a HAM-A score ≥29, a HAM-A score ≥30, a HAM-A score ≥32, a HAM-A score ≥34, a HAM-A score ≥36, a HAM-A score ≥38, a HAM-A score ≥40, a HAM-A score ≥42, a HAM-A score ≥44, a HAM-A score ≥46, a HAM-A score ≥48, and/or a HAM-A score ≥50 indicates moderate-to-severe anxiety. For example, a subject with a HAM-A score (e.g., a baseline HAM-A score) ≥15, a HAM-A score ≥17, a HAM-A score ≥19, a HAM-A score ≥21, a HAM-A score ≥23, a HAM-A score ≥25, a HAM-A score ≥29, a HAM-A score ≥30, a HAM-A score ≥32, a HAM-A score ≥34, a HAM-A score ≥36, a HAM-A score ≥38, a HAM-A score ≥40, a HAM-A score ≥42, a HAM-A score ≥44, a HAM-A score ≥46, a HAM-A score ≥48, and/or a HAM-A score ≥50 (e.g., a baseline HAM-A score ≥17) can be identified or diagnosed as having moderate-to-severe anxiety.

### Sheehan Disability Scale (SDS) Score

The Sheehan Disability Scale (SDS) score is an unweighted composite of three self-reported items of family, work and social impairment in the previous week. Each item is preceded by a lead-in question: “The symptoms have disrupted your (work/studies; social life; family life/home responsibilities)”. Each domain is scored from 11 potential responses ranging from (0)=not at all; (1-3)=mildly; (4-6)=moderately; (7-9)=markedly; and (10)=extremely, i.e., higher scores correspond to greater disruption. See, e.g., Sheehan, et al., Int. Clin. Psychopharmacol. Suppl 3:89-95 (1996) and Sheehan and Sheehan, Int. Clin. Psychopharmacol. 23: 70-83 (2008).

Work is specified as paid work, unpaid volunteer work, or training and subjects have the option to skip this item if they have not worked/or studied at all in the last week for reasons unrelated to their disorder, for example, normal retirement. Subscale scores for work, social, and family disability are calculated separately. The three domains can be summarized to evaluate global functional impairment by adding the scores of each of the three domains, resulting in global SDS score ranges from (0)=unimpaired to (30)=highly impaired.

### Columbia Suicide Severity Rating Scale (C-SSRS)

The Columbia Suicide Severity Rating Scale (C-SSRS) is a measure used to identify and assess individuals at risk for suicide. Questions can be administered in an interview/clinical setting or answers can be self-reported. The C-SSRS measures four constructs: the severity of ideation, the intensity of ideation, behavior and lethality.

The CSSRS provides several questions directed to suicidal ideation that the subject answers with a “yes” or a “no.” Such questions are directed to a wish to be dead; non-specific active suicidal thoughts; active suicidal ideation with any methods (not a plan) without intent to act; active suicidal ideation with some intent to act; without a specific plan; and active suicidal ideation with specific plan and intent. Additionally, the CSSRS includes features that are rated by the subject to help assess the intensity of ideation. Such features include asking about the frequency (e.g., less than one a week, once a week, 2-5 times a week, daily or almost daily, and many times each day); duration (e.g., fleeting, less than an hour, 1-4 hours, 4-8 hours, more than 8 hours); controllability (e.g., easily able to control thoughts, can control thoughts with little difficulty, can control thoughts with some difficulty, can control thoughts with a lot of difficulty, unable to control thoughts, and does not attempt to control thoughts); deterrents (e.g., deterrents definitely stopped you from attempting suicide, deterrents probably stopped you, uncertain deterrent stopped you, deterrent most likely did not stop you, and deterrents definitely did not stop you); and reasons for ideation (e.g., completely to get attention, mostly to get attention, equally to get attention and to end/stop pain, mostly to end/stop pain, and completely to end/stop pain). The CSSRS can also include questions directed to suicidal behavior and an actual suicide attempt such as asking about if an attempt was made; asking if anything was done to cause harm to one's self, and asking if the subject has done anything dangerous where he or she could have died. Each “yes” answer is 1 and each “no” answer is 0 for a total score of 0-10, with higher scores indicating increased suicidal ideation.

### Clinical Opiate Withdrawal Scale (COWS)

The Clinical Opiate Withdrawal Scale (COWS) is a clinician-administered that rates eleven opiate withdrawal symptoms. The summed score for the complete scale can be used to help clinicians determine the stage or severity of opiate withdrawal and assess the level of physical dependence on opioids. See, e.g., Wesson and Ling, J. Psychoactive Drugs, 35(2):253-9 (2003).

The eleven items in COWS include: (a) resting pulse rate, (measured as 0 (≤80 BPM), 1, 2, 4); (b) sweating (measured as 0 (no report of chills or flushing), 1, 2, 3, 4); (c) restlessness (measured as 0 (able to sit still), 1, 3, 5); (d) pupil size (measured as 0 (pupils pinned or normal size for room light), 1, 2, 5); (e) bone or joint aches (measured as 0 (not present), 1, 2, 4); (f) runny nose or tearing (measured as 0 (not present), 1, 2, 4); (g) GI upset (measured as 0 (no GI symptoms), 1, 2, 3, 5); (h) tremor (measured as 0 (no tremor), 1, 2, 4); (i) yawning (measured as 0 (no yawning), 1, 2, 4); (j) anxiety or irritability (measured as 0 (none), 1, 2, 4); (k) gooseflesh skin (measured as 0 (skin is smooth), 3, 5).

The score for each item reflects the severity of the sign or symptom. Total scores of 5-12 indicate mild withdrawal; total scores of 13-24 indicate moderate withdrawal; total scores of 25-36 indicate moderately severe withdrawal; and total scores of more than 36 indicate severe withdrawal.

### Montgomery Asberg Depression Rating Scale (MADRS)

The Montgomery-Åsberg Depression Rating Scale (MADRS) is a diagnostic questionnaire that can be used to measure the severity of a depressive episode in a subject. In some embodiments, the MADRS can be used to measure suicidal ideation. For example, the MADRS includes 10 items directed to the following: 1) apparent sadness (e.g., representing despondency, gloom and despair that is more than just ordinary transient low spirits that is reflected in speech, facial expression, and posture); 2) reported sadness (e.g., representing reports of depressed mood, regardless of whether it is reflected in appearance or not and can include low spirits, despondency or the feeling of being beyond help and without hope); 3) inner tension (e.g., representing feelings of ill-defined discomfort, edginess, inner turmoil, mental tension mounting to either panic, dread or anguish); 4) reduced sleep (e.g., representing the experience of reduced duration or depth of sleep compared to the subject's own normal pattern when well); 5) reduced appetite (e.g., representing the feeling of a loss of appetite compared with when-well); 6) concentration difficulties (e.g., representing difficulties in collecting one's thoughts mounting to an incapacitating lack of concentration); 7) lassitude (e.g., representing difficulty in getting started or slowness in initiating and performing everyday activities); 8) inability to feel (e.g., representing the subjective experience of reduced interest in the surroundings, or activities that normally give pleasure, and the ability to react with adequate emotion to circumstances or people is reduced); 9) pessimistic thoughts (e.g., representing thoughts of guilt, inferiority, self-reproach, sinfulness, remorse and ruin); and 10) suicidal thoughts (e.g., representing the feeling that life is not worth living, that a natural death would be welcome, suicidal thoughts, and preparations for suicide). Each item is rated from 0 to 6, with 0 reflecting that the subject is not at all as described by the item and 6 reflecting that the subject is extremely like what is described by the item. For example, for apparent sadness, a score of 0 can indicate that the subject does not display any sadness, whereas a score of 6 can indicate that the subject looks miserable all the time, e.g., the subject is extremely despondent. As another example, for suicidal thoughts, a score of 0 can indicate that the subject enjoys life or takes it as it comes; a score of 2 can indicate that the subject is weary of life and may have fleeting suicidal thoughts; a score of 4 can indicate that the subject feels he or she would probably be better off dead (e.g., suicidal thoughts are common, and suicide is considered as a possible solution, but without specific plans or intention); and a score of 6 can indicate that the subject has explicit plans for suicide when there is an opportunity (e.g., the subject has made active preparations for suicide).

Thus, the total score, after summation of each score for each item, is on a scale of 0 to 60. In some embodiments, a total score on the MADRS of about 0 to about 6 for the subject reflects the subject does not have symptoms related to depression; a score of about 7 to about 9 reflects that the subject has mild depression; a score of about 20 to about 34 reflects that the subject has moderate depression; and a score of about 34 to about 60 reflects that the subject has severe depression. MADRS is a clinician-rated scale.

In some embodiments, a MADRS total score (e.g., baseline MADRS total score) of about 20 to 60, about 21 to 60, about 22 to 60, about 23 to 60, about 24 to 60, about 25 to 60, about 30 to 60, about 32 to 60, about 34 to 60, about 36 to 60, about 38 to 60, about 40 to 60, about 42 to 60, about 44 to 60, about 46 to 60, about 48 to 60, about 50 to 60, about 52 to 60, about 54 to 60, about 56 to 60, and/or about 58 to 60 indicates moderate-to-severe anxiety. For example, a subject with a MADRS total score (e.g., baseline MADRS total score) of about 20 to 60, about 21 to 60, about 22 to 60, about 23 to 60, about 24 to 60, about 25 to 60, about 30 to 60, about 32 to 60, about 34 to 60, about 36 to 60, about 38 to 60, about 40 to 60, about 42 to 60, about 44 to 60, about 46 to 60, about 48 to 60, about 50 to 60, about 52 to 60, about 54 to 60, about 56 to 60, and/or about 58 to 60 can be identified or diagnosed as having moderate-to-severe anxiety. In some embodiments, a MADRS total score (e.g., baseline MADRS total score) of about 20 to 34, about 21 to 34, about 22 to 34, about 23 to 34, about 24 to 34, about 25 to 34, about 27 to 34, about 29 to 34, and/or about 31 to 34 indicates moderate-to-severe anxiety. For example, a subject with a a MADRS total score (e.g., baseline MADRS total score) of about 20 to 34, about 21 to 34, about 22 to 34, about 23 to 34, about 24 to 34, about 25 to 34, about 27 to 34, about 29 to 34, and/or about 31 to 34 can be identified or diagnosed as having moderate-to-severe anxiety. In some embodiments, a MADRS total score (e.g., baseline MADRS total score) of about 34 to 60, about 36 to 60, about 38 to 60, about 40 to 60, about 42 to 60, about 44 to 60, about 46 to 60, about 48 to 60, about 50 to 60, about 52 to 60, about 54 to 60, about 56 to 60, and/or about 58 to 60 indicates moderate-to-severe anxiety. For example, a subject with a MADRS total score (e.g., baseline MADRS total score) of about 34 to 60, about 36 to 60, about 38 to 60, about 40 to 60, about 42 to 60, about 44 to 60, about 46 to 60, about 48 to 60, about 50 to 60, about 52 to 60, about 54 to 60, about 56 to 60, and/or about 58 to 60 can be identified or diagnosed as having moderate-to-severe anxiety. In some embodiments, a MADRS total score (e.g., baseline MADRS total score) of ≥25 (e.g., about 25 to 34, about 27 to 34, about 29 to 34, about 31 to 34, about 33 to 34, about 25 to 60, about 26 to 60, about 27 to 60, about 28 to 60, about 29 to 60, about 30 to 60, about 32 to 60, about 34 to 60, about 36 to 60, about 38 to 60, about 40 to 60, about 42 to 60, about 44 to 60, about 46 to 60, about 48 to 60, about 50 to 60, about 52 to 60, about 54 to 60, about 56 to 60, and/or about 58 to 60) indicates moderate-to-severe anxiety. For example, a subject with a MADRS total score (e.g., baseline MADRS total score) of ≥25 (e.g., about 25 to 34, about 27 to 34, about 29 to 34, about 31 to 34, about 33 to 34, about 25 to 60, about 26 to 60, about 27 to 60, about 28 to 60, about 29 to 60, about 30 to 60, about 32 to 60, about 34 to 60, about 36 to 60, about 38 to 60, about 40 to 60, about 42 to 60, about 44 to 60, about 46 to 60, about 48 to 60, about 50 to 60, about 52 to 60, about 54 to 60, about 56 to 60, and/or about 58 to 60) can be identified or diagnosed as having moderate-to-severe anxiety.

### Clinical Global Impression—Severity (CGI-S)

The Clinical Global Impression—Severity (CGI-S) Scores is a subscale of Clinical Global Impression (CGI), a retrospective assessment that is a measure of symptom severity, treatment response and the efficacy of treatments in treatment studies, completed by the treating physician at baseline and at subsequent clinic visits to document any change in target symptoms documented at baseline. The CGI-S evaluates the presence of relevant symptoms, the frequency of their occurrence over the seven day rating timeframe, the intensity or severity of the symptoms, and the effect of the symptoms on functioning in major areas of the patient's life—work, home, school, and relationships. The typical time span rated for severity of illness is now or within the last week. See, e.g., Busner and Targum, Psychiatry, 4(7), 28-37 (2007).

The CGI-S asks the clinician one question: “Considering your total clinical experience with this particular population, how mentally ill is the patient at this time?” which is rated on the following seven-point scale: (1)=normal, not at all ill (symptoms of disorder not present past seven days); (2)=borderline mentally ill (subtle or suspected pathology); (3)=mildly ill (clearly established symptoms with minimal, if any, distress or difficulty in social and occupational function); (4)=moderately ill (overt symptoms causing noticeable, but modest, functional impairment or distress; symptom level may warrant medication); (5)=markedly ill (intrusive symptoms that distinctly impair social/occupational function or cause intrusive levels of distress); (6)=severely ill (disruptive pathology, behavior and function are frequently influenced by symptoms, may require assistance from others); (7)=among the most extremely ill patients (pathology drastically interferes in many life functions; may be hospitalized).

This rating is based upon observed and reported symptoms, behavior, and function in the past seven days. As symptoms and behavior can fluctuate over a week; the score should reflect the average severity level across the seven days.

### Symptoms of Major Depressive Disorder Scale (SMDDS)

The Symptoms of Major Depressive Disorder Scale (SMDDS) is a patient-reported outcome measure based on a 16-item scale addressing nine different domains of major depressive disorder (MDD) over the last seven days: (1) negative emotions/mood (4 items: sadness, hopeless/helpless, irritability, difficulty enjoying daily life (anhedonia); (2) anxiety (2 items: feeling overwhelmed, worry); (3) low energy (1 item: tiredness); (4) cognition (2 items: intrusive thoughts, poor concentration); (5) sleep disturbances (1 item: general sleep adequacy); (6) self-harm/suicide (1 item: life not worth living); (7) low motivation (2 items: lack of drive, no interest in activities); (8) sense-of-self (1 item: blame); and (9) eating behavior (2 items, scored as a single item: poor appetite, over eating). See, e.g., Bushnell, et al., Value Health, 22(8):906-915 (2019).

The SMDDS assesses changes in depressive symptom severity for adults (aged 18 years or older) who have been diagnosed and are being treated in an ambulatory setting for MDD. Because the symptom experience of MDD is chronic, the SMDDS measure asks respondents to report on the status of their MDD symptoms over the past seven days. Each item requires a response on a 5-point verbal rating scale using either (0)=not at all, (1)=a little bit, (2)=moderately, (3)=quite a bit, (4)=extremely (for intensity items); or (0)=never, (1)=rarely, (2)=sometimes, (3)=often, (4)=always (for frequency items). Higher scores indicate greater severity of MDD symptomology, with a total score range of 0-60.

### Self-Assessment of Treatment Experience (SATE) Score

The Self-Assessment of Treatment Experience (SATE) is a questionnaire with a one or two item self-report scale which provides additional information regarding an individual's subjective experience on depression, often since starting a new medication. This is a qualitative scale and the responses will be recorded as ‘very much improved’, ‘much improved’, ‘improved’, ‘no change’, ‘worse’, ‘much worse’, and ‘very much worse’.

### Hamilton Anxiety Scale 6 (HAM-A6)

The Hamilton Anxiety Scale 6 (HAM-A6) is a clinician-rated scale including the 6-item Hamilton Anxiety Scale (HAM-A) subscale, which scale assesses the severity of different anxiety-related symptoms with a score range of 0 to 52. Each item is rated on a 5-point scale ranging from 0=not present; 1=mild degree; 2=moderate degree; 3=marked degree; 4=maximum degree. Each of the 14 items is rated by the clinician on a 5-point scale ranging from 0 (not present) to 4 (maximum degree). The 6 item subscale from HAM-A (HAM-A6) is a uni-dimensional, 6-item subscale derived from the original HAM-A. The HAM-A6 comprises of five psychic anxiety symptoms: anxious mood, psychic tension, fears, intellectual disturbances, and anxious behavior observed at the interview, as well as one somatic item, muscular tension, with a score range of 0 to 24. Higher scores represent more severe anxiety symptoms. See, e.g., Kent, et al., Prog. Neuropsychopharmacol. Biol. Psychiatry, 67:66-73 (2016) and Meoni, et al., J. Clin. Psychiatry, 62(11): 888-893 (2001).

### Structured Interview Guide for the Hamilton Anxiety Rating Scale (SIGH-A)

The Structured Interview Guide for the Hamilton Anxiety Rating Scale (SIGH-A) Score is a clinician-rated 14-item scale used to measure severity of different anxiety-related symptoms in subjects. Each of the 14 items is rated by the clinician on a 5-point scale ranging from 0 (not present) to 4 (maximum degree) with a total score range of 0 to 52 where higher score indicates worsening. Much like the HARS, the scale in SIGH-A relates to: (1) anxious mood; (2) tension; (3) fears; (4) insomnia; (5) ‘intellectual’; (6) depressed mood; (7) somatic symptoms; (8) sensory; (9) cardiovascular; (10) respiratory; (11) gastrointestinal; (12) genitourinary; (13) autonomic; and (14) observed behavior at interview. See, e.g., Shear, et al., Depress. Anxiety, 13(4): 166-78 (2001) and Rollman, et al., Arch. Gen. Psychiatry, 62(12): 1332-41 (2005).

### Prior Administration of Other Therapeutic Agents

In some embodiments, the subject was previously administered one or more therapeutic agents, i.e., antidepressant agents, prior to the administration of 1-(6-ethyl-8-fluoro-4-methyl-3-(3-methyl-1,2,4-oxadiazol-5-yl)quinolin-2-yl)-N-(tetrahydro-2H-pyran-4-yl)piperidin-4 amine, or a pharmaceutically acceptable salt thereof.

Some embodiments reference a time “prior to” administration of 1-(6-ethyl-8-fluoro-4-methyl-3-(3-methyl-1,2,4-oxadiazol-5-yl)quinolin-2-yl)-N-(tetrahydro-2H-pyran-4-yl)piperidin-4 amine, or a pharmaceutically acceptable salt thereof. The time prior to administration may be a particular time or range of time as indicated (e.g., about 30 minutes, about 1 hour, from about 1 day to about 1 week, 6 months, etc.), or it may be any time prior to administration if no particular time or range is specified.

In some embodiments, the subject has previously been administered a standard of care treatment for depression (including major depressive disorder) and the subject was not responsive to the previous therapy.

In some embodiments, the subject has previously been administered a standard of care treatment for anhedonia and the subject was not responsive to the previous therapy.

In some embodiments, the subject has previously been administered one or more antidepressants, and was not responsive to the previous therapy.

In some embodiments, the subject has previously been administered one or more antidepressants and was not responsive to the previous therapy. In some embodiments, the antidepressant is an atypical antidepressant, a selective serotonin reuptake inhibitor, a selective serotonin and norepinephrine reuptake inhibitor, a monoamine oxidase inhibitor, a kappa opioid receptor antagonist, or a selective norepinephrine reuptake inhibitor, and was not responsive to the previous therapy.

In some embodiments, the subject has previously been administered one or more selective serotonin reuptake inhibitors, such as citalopram, escitalopram, fluoxetine, fluvoxamine, paroxetine, and sertraline, and was not responsive to the previous therapy.

In some embodiments, the subject has previously been administered one or more selective serotonin and norepinephrine reuptake inhibitors, such as atomoxetine, desvenlafaxine, duloxetine, levomilnacipran, milnacipran, sibutramine, tramadol, and venlafaxine, and was not responsive to the previous therapy.

In some embodiments, the subject has previously been administered one or more monoamine oxidase inhibitors, such as moclobemide, rasagiline, selegiline, or safinamide, and was not responsive to the previous therapy.

In some embodiments, the subject has previously been administered one or more selective norepinephrine reuptake inhibitors, such as reboxetine, and was not responsive to the previous therapy.

In some embodiments, the subject has previously been administered one or more benzodiazepines, such as alprazolam, bromazepam, chlordiazepoxide, clonazepam, clorazepate, diazepam, flurazepam, lorazepam, oxazepam, temazepam, or triazolam, and was not responsive to the previous therapy.

In some embodiments, the subject has previously been administered one or more a kappa opioid receptor antagonists and was not responsive to the previous therapy. In some embodiments, the kappa opioid receptor antagonist is aticaprant.

In some embodiments, the subject exhibited no clinically meaningful response to a prior standard of care treatment for depression, as described herein.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1. Effects of 1-[6-ethyl-8-fluoro-4-methyl-3-(3-methyl-1,2,4-oxadiazol-5-yl)quinolin-2-yl]-N-(oxan-4-yl)piperidin-4-amine on Symptoms of Major Depressive Disorder in a Phase 3 Trial

### Introduction

A significant unmet need remains in the treatment of Major Depressive Disorder (MDD), as many patients do not adequately respond to approved pharmacotherapies and often experience residual symptoms and intolerable side effects (1-3). Anhedonia and anxiety are common presenting symptoms that are linked to more severe MDD and poorer response to antidepressant treatment (4-6). In MDD patients with anxiety, remission rates are lower, and remission takes longer to achieve than in those without anxiety (7, 8). The kappa opioid receptor (KOR)/dynorphin system is a well-characterized pathway, and results from preclinical and clinical studies support its potential to modulate depression, anhedonia, and anxiety (9-11).

1-[6-ethyl-8-fluoro-4-methyl-3-(3-methyl-1,2,4-oxadiazol-5-yl)quinolin-2-yl]-N-(oxan-4-yl)piperidin-4-amine (also referred to herein as “the Study Compound”, “the study drug”, “navacaprant”, “NMRA-140”, and/or “BTRX-335140”) is a novel, oral, once-daily, highly selective KOR antagonist in development as monotherapy for the treatment of MDD. In three replicate Phase 3 randomized, placebo-controlled, double-blind studies, KOASTAL-1, KOASTAL-2, and KOASTAL-3, the efficacy and safety of navacaprant monotherapy in adult patients with moderate-to-severe MDD was evaluated.

### KOASTAL Program

The KOASTAL program includes three replicate Phase 3 randomized, placebo-controlled, double-blind studies, KOASTAL-1, KOASTAL-2, and KOASTAL-3, designed to evaluate the efficacy and safety of navacaprant monotherapy in adult patients with moderate-to-severe MDD who have a MADRS total score ≥25 at baseline. The KOASTAL-1 Study was conducted in the U.S. The KOASTAL-2 and -3 studies include sites in the U.S. and other regions. The primary endpoint of these studies is change from baseline in MADRS total score at Week 6. Key secondary endpoints include change from baseline on the SHAPS at Week 6, a measure of anhedonia.

The KOASTAL Program also includes an open-label extension study, KOASTAL-LT, designed to evaluate the long-term safety of navacaprant. A significant portion of patients who received navacaprant 80 mg (83.3%) in the KOASTAL-1 study elected to enroll in KOASTAL-LT. Patients will also have the opportunity to enroll in the KOASTAL-LT study following participation in the KOASTAL-2 and KOASTAL-3 studies.

### KOASTAL-1 Study Summary Results

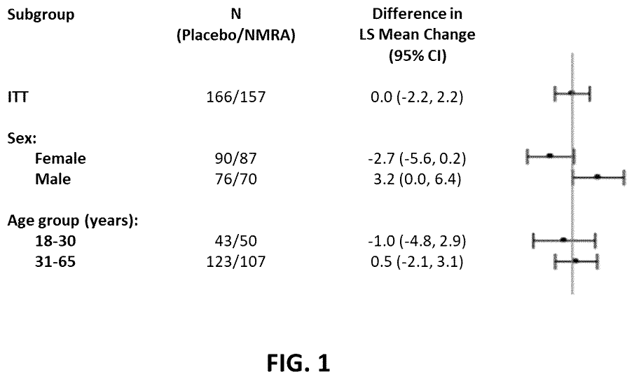

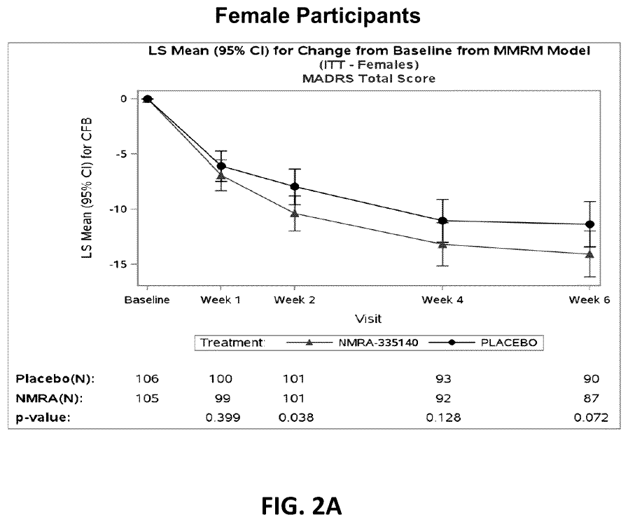

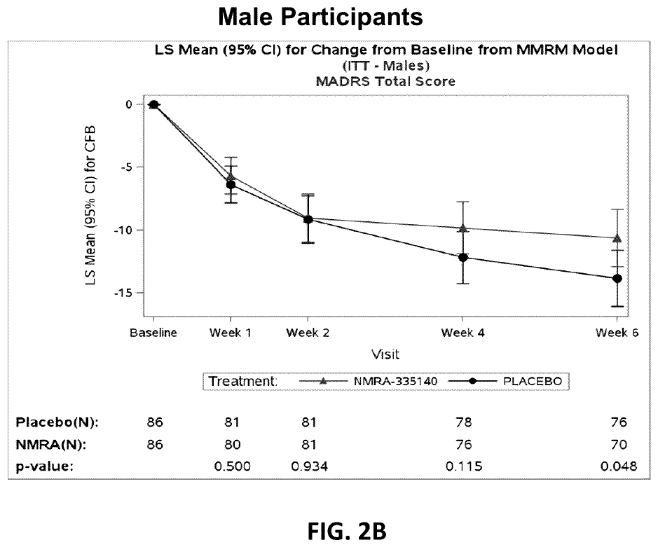

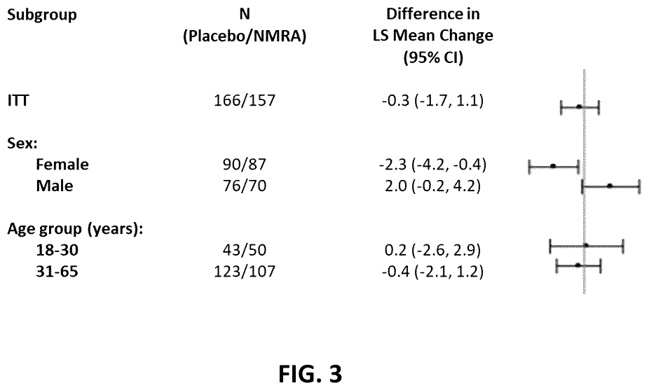

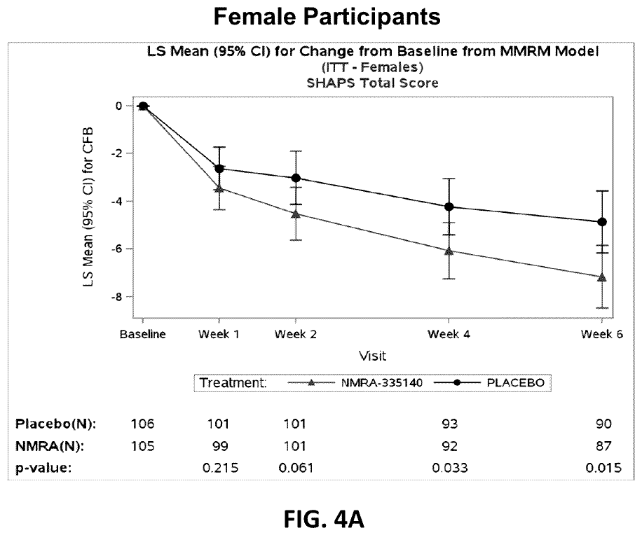

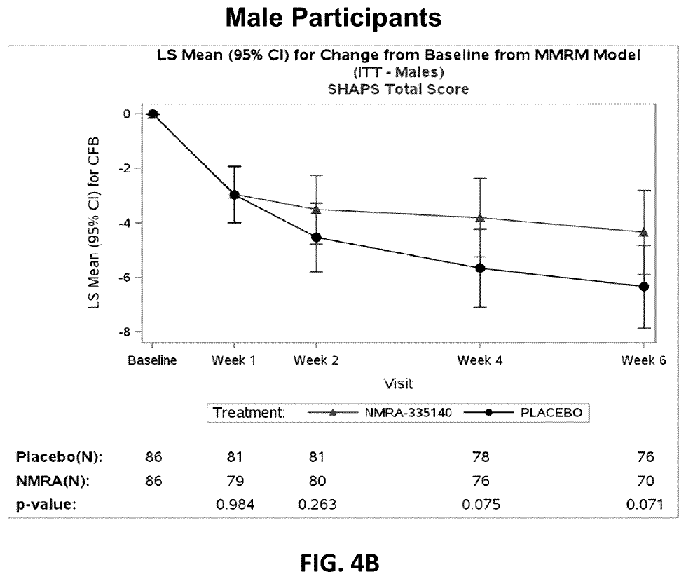

Topline efficacy results for navacaprant compared to placebo are outlined in Table 1 and described in FIG. 1, FIGS. 2A and 2B, FIG. 3, and FIGS. 4A and 4B.

TABLE 1
Efficacy Results for Navacaprant

| | | | | |
|---|---|---|---|---|
| | Navacaprant | | LS Mean | p |
| Outcome | 80 mg | Placebo | Difference | value |

MADRS Total Score

| | | | | |
|---|---|---|---|---|
| ITT population at Week 6 | −12.5 | −12.5 | 0 | 0.933 |
| (Primary Endpoint) | (n = 191) | (n = 192) | | |
| Female population at | −14.0 | −11.4 | −2.7 | 0.072 |
| Week 6 | (n = 105) | (n = 106) | | |
| Male population at Week 6 | −10.6 | −13.8 | 3.2 | 0.048 |
| | (n = 86) | (n = 86) | | |

SHAPS Total Score

| | | | | |
|---|---|---|---|---|
| ITT population at Week 6 | −5.7 | −5.5 | −0.3 | 0.648 |
| (Key Secondary Endpoint) | (n = 191) | (n = 192) | | |
| Female population at | −7.2 | −4.9 | −2.3 | 0.015 |
| Week 6 | (n = 105) | (n = 106) | | |
| Male population at Week 6 | −4.3 | −6.3 | 2.0 | 0.071 |
| | (n = 86) | (n = 86) | | |

Results for other secondary efficacy endpoints are outlined in Table 2 and Table 3.

TABLE 2
Results for Other Secondary Efficacy Endpoints

| | SS for | | | Between- | | |
|---|---|---|---|---|---|---|
| | CFB at | | | group | | |
| Endpoint | Week 6 | | | Difference | | |
| (N1/N2, SS at Baseline) | (n₁/n₂) | Placebo | Navacaprant | (SE) | 95% CI | p-value |
| SDS total score CFB at | 169/163 | −7.2 | −7.1 (0.6) | 0.0 (0.8) | (−1.5, 1.6) | 0.982 |
| Week 6 (192/190) | | (0.6) | | | | |
| CGI-S CFB at Week 6 | 164/155 | −1.4 | −1.3 (0.1) | 0.0 (0.1) | (−0.3, 0.3) | 0.946 |
| (190/189) | | (0.1) | | | | |
| CGI-I at Week 6 | 164/155 | 2.7 | 2.7 (0.1) | 0.0 (0.1) | (−0.2, 0.3) | 0.862 |
| | | (0.1) | | | | |
| HAM-A total score CFB at | 166/157 | −5.3 | −5.2 (0.4) | 0.1 (0.6) | (−1.1, 1.3) | 0.900 |
| Week 6 (192/190) | | (0.4) | | | | |

Note:
difference is calculated as navacaprant − placebo. Negative difference implies the benefit of navacaprant vs. placebo.
Note:
for CGI-S, SDS total score and HAMA total score, the results are LS mean change from baseline to Week 6 with SE; for CGI-I the results are LS mean at Week 6; SS = sample size.

TABLE 3
Results for Other Secondary Efficacy Endpoints (cont.)

| | | | Between- | | |
|---|---|---|---|---|---|
| Endpoint (m1/m2, SS | n/m (%) of Responders | | group | | |
| at Baseline) | Placebo | Navacaprant | Difference | 95% CI | p-value |
| Response Rate at Week 6 | 58/192 | 58/191 | 0.2% | (−9.0%, | 0.973 |
| (primary) (192/191) | (30.2%) | (30.4%) | | 9.3%) | |
| Response Rate at Week 6 | 58/166 | 58/157 | 2.0% | (−8.4%, | 0.709 |
| (observed case) (166/157) | (34.9%) | (36.9%) | | 12.4%) | |
| Remission Rate at Week 6 | 36/192 | 38/191 | 1.1% | (−6.7%, | 0.778 |
| (primary) (192/191) | (18.8%) | (19.9%) | | 9.0%) | |
| Remission Rate at Week 6 | 36/166 | 38/157 | 2.6% | (−6.6%, | 0.586 |
| (observed case) (166/157) | (21.7%) | (24.2%) | | 11.7%) | |

Note:
difference is calculated as navacaprant − placebo. Positive difference implies the benefit of navacaprant vs. placebo; SS = sample size.

Navacaprant was shown to be safe and generally well-tolerated with no serious adverse events reported. There was no signal for increased suicidal ideation or suicidal behavior compared to placebo, as measured by Columbia Suicide Severity Rating Scale (C-SSRS).

LIST OF ABBREVIATIONS

| Abbreviation | |
|---|---|
| or Term | Definition |
| Ab | Antibody |
| ADD | attention deficit disorder |
| ADHD | attention deficit hyperactivity disorder |
| AE | adverse event |
| ALT | alanine aminotransferase |
| APA | American Psychiatric Association |
| AESI(s) | adverse event(s) of special interest |
| AST | aspartate aminotransferase |
| ANCOVA | analysis of the covariance |
| AUD | alcohol use disorder |
| BMI | body mass index |
| BP | blood pressure |
| CAT | Clinical Assessment Technology |
| CFR | Code of Federal Regulations |
| cGCP | current Good Clinical Practice |
| CGI-I | Clinical Global Impression of Improvement |
| CGI-S | Clinical Global Impression of Severity |
| CI | confidence interval |
| CIOMS | Council for International Organizations of |
| | Medical Sciences |
| CKD-EPI | Chronic Kidney Disease Epidemiology Collaboration |
| CNS | central nervous system |
| COVID-19 | coronavirus disease 2019 |
| COWS | Clinical Opiate Withdrawal Scale |
| CREB | cAMP response binding protein |
| CRF | case report form |
| CRO | Contract Research Organization |
| C-SSRS | Columbia Suicide Severity Rating Scale |
| CYP | cytochrome P450 |
| DSM-5 | Diagnostic and Statistical Manual of Mental |
| | Disorders Fifth Edition |
| DSM-IV-TR | Diagnostic and Statistical Manual of Mental |
| | Disorders, Fourth Edition, Text Revision |
| EC | Ethics Committee |
| ECG | electrocardiogram, electrocardiographic |
| eCRF | electronic case report form |
| EDC | electronic data capture |
| eGFR | estimated glomerular filtration rate |
| FDA | Food and Drug Administration |
| GAD | generalized anxiety disorder |
| GEE | generalized estimating equations |
| HADS | Hospital Anxiety and Depression Scale |
| HADS-A | Hospital Anxiety and Depression Scale - |
| | Anxiety Subscale |
| HADS-D | Hospital Anxiety and Depression Scale - |
| | Depression Subscale |
| HAM-A | Hamilton Anxiety Rating Scale |
| HAMD-17 | Hamilton Depression Rating Scale - |
| | 17-item Version |
| HAV | hepatitis A virus |
| HAV-Ab (IgM) | hepatitis A virus antibody |
| HBs Ag | hepatitis B surface antigen |
| HCV | hepatitis C virus |
| HIPAA | Health Insurance Portability and |
| | Accountability Act |
| HIV | human immunodeficiency virus |
| HPMC | hydroxypropyl methylcellulose |
| IC₅₀ | half-maximal inhibitory concentration |
| ICC | intraclass correlation coefficient |
| ICF | informed consent form |
| ICH | International Council for Harmonisation of |
| | Technical Requirements for Pharmaceuticals |
| | for Human Use |
| IEC | Independent Ethics Committee |
| IgM | immunoglobulin M |
| IND | US Investigational New Drug Application |
| IRB | Institutional Review Board |
| IWRS | interactive web-response system |
| KOR | kappa opioid receptor |
| KORA | kappa opioid receptor antagonist |
| LAC | acetyl-L-carnitine |
| LC-MS/MS | liquid chromatography-tandem mass spectrometry |
| LLN | lower limit of normal |
| LS Means | least-square means |
| MedDRA | Medical Dictionary for Regulatory Activities |
| MDD | major depressive disorder |
| MMRM | mixed-models repeated measures |
| NOAEL | no-observed-adverse-effect-level |
| OPRK1 | opioid receptor kappa 1 |
| PDF | portable document format |
| PET | positron emission tomography |
| P-gp | P-glycoprotein |
| PHQ-9 | Patient Health Questionnaire-9 |
| PK | pharmacokinetic(s) |
| POC | proof of concept |
| PP | per-protocol |
| PRT | probabilistic reward task |
| PTSD | post-traumatic stress disorder |
| QD | once daily |
| QTcF | Fridericia's correction method for QT Interval |
| REB | Research Ethics Board |
| RO | receptor occupancy |
| S-anxiety | STAI state anxiety subscale score |
| SAE | serious adverse event |
| SAP | statistical analysis plan |
| SCID-5-CT | Structured Clinical Interview for DSM-5 Disorders, |
| | Clinical Trials Version |
| SD | standard deviation |
| SDS | Sheehan Disability Scale |
| SHAPS | Snaith-Hamilton Pleasure Scale |
| SIGH-A | Structured Interview Guide for the Hamilton |
| | Anxiety Rating Scale |
| SIGH-D | Structured Interview Guide for the Hamilton |
| | Depression Rating Scale |
| SNRI | serotonin norepinephrine reuptake inhibitor |
| SOP | Standard Operating Procedure |
| SSRI | selective serotonin reuptake inhibitor |
| STAI | State-Trait Anxiety Inventory |
| SUSAR | serious unexpected suspected adverse reaction |
| T-anxiety | STAI trait anxiety subscale score |
| TC | telephone call |
| TEAE | treatment-emergent adverse event |
| THC | Tetrahydrocannabinol |
| TPO | third-party organization |
| TSH | thyroid-stimulating hormone |
| UDS | urine drug screen |
| ULN | upper limit of normal |
| US | United States |
| WHO | World Health Organization |
| WHODrug | World Health Organization Drug classification |

A number of embodiments of the present disclosure have been described. Nevertheless, it will be understood that various modifications may be made without departing from the spirit and scope of the disclosure. Accordingly, other embodiments are within the scope of the following claims.

1. A method of treating depression in a female subject previously identified or diagnosed as having moderate-to-severe anxiety or as having anhedonia and moderate-to-severe anxiety, the method comprising administering to the female subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

2.-3. (canceled)

4. The method of claim 1, wherein the method further comprises identifying the female subject as having one or more symptoms of anhedonia.

5.-15. (canceled)

16. The method of claim 1, wherein the depression is major depressive disorder (MDD) or treatment resistant depression (TRD).

17.-18. (canceled)

19. The method of claim 1, wherein the female subject has a baseline Montgomery-Åsberg Depression Rating Scale (MADRS) total score ≥25.

20. A method of treating severe depression in a female subject in need thereof, comprising administering to the female subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the female subject has been previously identified as having a Montgomery-Åsberg Depression Rating Scale (MADRS) total score of from 25 to 34, or as having moderate-to-severe anxiety and a Montgomery-Åsberg Depression Rating Scale (MADRS) total score of from 25 to 34.

21. (canceled)

22. The method of claim 20, wherein the MADRS total score of the female subject is decreased after four weeks or after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

23. (canceled)

24. The method of claim 20, wherein the female subject has a baseline MADRS total score ≥25.

25. A method of treating depression in a female subject previously identified or diagnosed as having moderate-to-severe anxiety, the method comprising:
(a) determining a first Montgomery-Åsberg Depression Rating Scale (MADRS) total score in the subject;
(b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and
(c) determining a second MADRS total score in the subject; wherein the second MADRS total score is less than the first MADRS total score.

26. (canceled)

27. The method of claim 25, wherein the period of time is about 1 week to about 52 weeks, about 4 weeks to about 26 weeks, about 4 weeks to about 12 weeks, or about 6 weeks.

28.-30. (canceled)

31. The method of claim 25, wherein the first MADRS total score is 25 to 60 or 25 to 34.

32. (canceled)

33. The method of claim 25, wherein the female subject has a baseline MADRS total score ≥25.

34. The method of claim 25, wherein the female subject has a Snaith-Hamilton Pleasure Scale (SHAPS) total score of 31 to 56 or 37 to 56 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

35. (canceled)

36. The method of claim 34, wherein the SHAPS total score of the female subject is decreased after four weeks or after six weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

37. (canceled)

38. The method of claim 1, wherein prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, the female subject has a baseline MADRS total score ≥25, a MADRS total score of about 25 to 60, or a MADRS total score of about 25 to 34.

39.-40. (canceled)

41. The method of claim 38, wherein the MADRS total score of the female subject is decreased after four weeks or after six weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

42. (canceled)

43. The method of claim 1, wherein the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is administered once daily for the period of time.

44. The method of claim 1, wherein the female subject has previously been administered one or more antidepressant medications.

45. The method of claim 44, wherein the female subject did not exhibit a clinically meaningful response to the one or more previously administered antidepressant medications.

46. The method of claim 44, wherein the one or more previously administered antidepressant medications are selected from selective serotonin reuptake inhibitors, selective norepinephrine reuptake inhibitors, and kappa opioid receptor antagonists.

47.-50. (canceled)

51. The method of claim 46, wherein the kappa opioid receptor antagonist is aticaprant.

52. The method of claim 1, wherein the female subject has not been previously administered one or more antidepressant medications.

53. (canceled)

54. The method of claim 1, wherein the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is from about 20 mg/day to about 160 mg/day, from about 20 mg/day to about 80 mg/day, from about 80 mg/day to about 120 mg/day, or from about 120 mg/day to about 160 mg/day.

55.-57. (canceled)

58. The method of claim 1, wherein the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is 40 mg/day, 80 mg/day, 120 mg/day, or 160 mg/day.

59.-61. (canceled)

62. The method of claim 1, wherein the female subject has not had a score of “YES” on C-SSRS Item 4 or Item 5 within 3 months prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

63. The method of claim 1, wherein the 1-[6-ethyl-8-fluoro-4-methyl-3-(3-methyl-1,2,4-oxadiazol-5-yl)quinolin-2-yl]-N-(oxan-4-yl)piperidin-4-amine is administered as the free base or as a pharmaceutically acceptable salt.

64. (canceled)
